(19) United States
(12) Patent Application Publication
(10) Pub. No.: US 2025/0213525 A1
(43) Pub. Date: Jul. 3, 2025

(54) Atopic Dermatitis Improving Agent, Interleukin Production Inhibitor, and Inhibitor of Expression of Interleukin Gene

(71) Applicant: PIAS CORPORATION, Osaka-shi (JP)

(72) Inventors: Shunya Sahara, Osaka-shi (JP); Miki Iwai, Osaka-shi (JP); Ayumi Ueno, Osaka-shi (JP); Koichi Nakaoji, Osaka-shi (JP); Kazuhiko Hamada, Osaka-shi (JP)

(21) Appl. No.: 18/851,716

(22) PCT Filed: Mar. 8, 2023
(86) PCT No.: PCT/JP2023/008804

§ 371 Date: Sep. 27, 2024

(30) Foreign Application Priority Data
Mar. 28, 2022 (JP) 2022-051537

(51) Int. Cl. A61K 31/375; A61P 37/06

(52) U.S. Cl. CPC A61K 31/375; A61P 37/06

(57) ABSTRACT

Provided are an atopic dermatitis improving agent and the like including a glyceryl ascorbic acid acylated derivative having a specific molecular structure.

### CROSS-REFERENCE TO RELATED APPLICATIONS

This application is the United States national phase of International Patent Application No. PCT/JP2023/008804 filed Mar. 8, 2023, and claims priority to Japanese Patent Application No. 2022-051537 filed Mar. 28, 2022, the disclosures of which are hereby incorporated by reference in their entireties.

### BACKGROUND OF THE INVENTION

### Field of the Invention

The present invention relates to an atopic dermatitis improving agent, an interleukin production inhibitor, and an inhibitor of expression of interleukin gene.

### Description of Related Art

Atopic dermatitis is a chronic disease in which itchy eczema is repeatedly exacerbated and improved, and this atopic dermatitis is thought to develop as a result of a complex interaction of various factors. Atopic dermatitis is thought to be exacerbated by, for example, the production of lipid mediators that induce allergic reactions, but it is thought that this is not the only cause of exacerbation of atopic dermatitis.

A variety of atopic dermatitis improving agents for improving the aforementioned atopic dermatitis has been conventionally known.

As this type of atopic dermatitis improving agent, there is known an atopic dermatitis improving agent including a docosahexaenoic acid (DHA)- and/or eicosapentaenoic acid (EPA)-containing ointment and a tacrolimus ointment (Patent Literature 1).

The atopic dermatitis improving agent described in Patent Literature 1 can inhibit the production of leukotriene B4 that is one of lipid mediators inducing allergic reactions, and thus can improve symptoms of atopic dermatitis.

### CITATION LIST

### Patent Literature

Patent Literature 1: JP 2017-197471 A

### SUMMARY OF THE INVENTION

### Technical Problem

Nevertheless, no sufficient consideration appears to have been made on a preparation that can improve atopic dermatitis.

It is therefore an object of the present invention to provide an atopic dermatitis improving agent, an interleukin production inhibitor, and an inhibitor of expression of interleukin gene, which can improve atopic dermatitis.

### Solution to Problem

An atopic dermatitis improving agent according to the present invention is characterized by including a glyceryl ascorbic acid acylated derivative represented by formula (I) below.

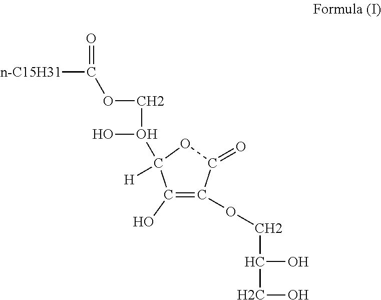

An interleukin production inhibitor according to the present invention includes the glyceryl ascorbic acid acylated derivative represented by formula (I) above and is configured to inhibit production of interleukin-33 and interleukin-1.

An inhibitor of expression of interleukin gene according to the present invention includes the glyceryl ascorbic acid acylated derivative represented by formula (I) above, and is configured to inhibit expression of interleukin-33 gene and interleukin-1 gene.

### BRIEF DESCRIPTION OF THE DRAWINGS

FIG. 1 is a graph showing test results (in vitro tests) of evaluating the influence on expression of interleukin-33 (IL-33) gene.

FIG. 2 is a graph showing test results (in vitro tests) of evaluating the influence on expression of interleukin-1α (IL-1α) gene.

FIG. 3 is a graph showing test results (in vitro tests) of evaluating the influence on expression of interleukin-1β (IL-1β) gene.

FIG. 4 is a graph showing test results (in vitro tests) of evaluating the influence on gene expression of interleukin-33 (IL-33).

FIG. 5 is a graph showing test results (in vitro tests) of evaluating the influence on expression of interleukin-1α (IL-1α) gene.

FIG. 6 is a graph showing test results (in vitro tests) of evaluating the influence on expression of interleukin-1β (IL-1β) gene.

### DESCRIPTION OF THE INVENTION

Hereinafter, a description will be given on one embodiment of an atopic dermatitis improving agent, an interleukin production inhibitor, and an inhibitor of expression of interleukin gene (hereinafter, also referred simply to as a preparation) according to the present invention.

The preparation of this embodiment includes a glyceryl ascorbic acid acylated derivative represented by formula (I) below.

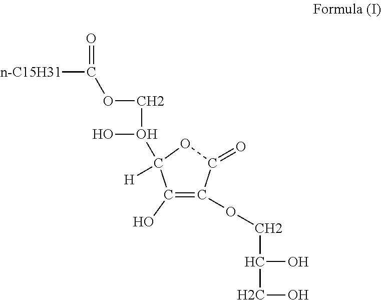

The preparation of this embodiment that includes the glyceryl ascorbic acid acylated derivative represented by formula (I) above enables to exhibit an inhibition effect on the production of a specific inflammatory cytokine related to atopic dermatitis, and thus can improve atopic dermatitis.

The compound represented by formula (I) above may be referred to as 2-O-glyceryl-6-O-hexadecanoyl ascorbic acid. Sufficient improvement of atopic dermatitis is not necessarily achieved by another compound similar to the compound represented by formula (I) above such as 2-O-glyceryl-6-ethanoyl ascorbic acid, 2-O-glyceryl-6-butanoyl ascorbic acid, 2-O-glyceryl-6-octanoyl ascorbic acid (2-O-glyceryl-6-(2-ethylhexanoyl) ascorbic acid), 2-O-glyceryl-6-tetradecanoyl ascorbic acid, 2-O-glyceryl-6-octadecanoyl ascorbic acid, or 2-O-glyceryl-6-docosanoyl ascorbic acid.

The preparation of this embodiment including the glyceryl ascorbic acid acylated derivative represented by formula (I) enables to inhibit the production of cytokine to inflammatory stimuli of epidermal cells. For example, it is possible to inhibit the production of a specific cytokine such as interleukin-33-(IL-33) or interleukin-1 (IL-1a or IL-1β). Thereby, it is possible to improve atopic dermatitis.

Interleukin 33-(IL-33) is a cytokine involved in pathological conditions of allergic diseases, and is a cytokine that exacerbates the pathological conditions of, in particular, atopic dermatitis among skin allergic diseases. Specifically, interleukin-33 (IL-33) is one of cytokines produced from epidermal cells and is excessively expressed in epidermal cells of a atopic dermatitis patient. More specifically, interleukin-33 (IL-33) activates group 2 innate lymphoid cells (ILC2s) and Th2 cells being helper T cells, and induces Th2 cytokines such as interleukin-4 (IL-4) and interleukin-13 (IL-13), thereby causing a Type 2 inflammatory response, mainly atopic dermatitis. Interleukin-1 (IL-1) is an inflammatory cytokine involved in a variety of inflammation and is a cytokine also related to exacerbation of atopic dermatitis. Interleukin-1 (IL-1) is, for example, produced by epidermal cells in response to various stimuli to cause inflammation. IL-1 receptors on mature helper T cells are present only on Th2 cells. IL-1 may cause proliferation of Th2 cells, which may lead to the onset or exacerbation of atopic dermatitis.

The preparation of this embodiment can inhibit expression of the aforementioned inflammatory cytokine gene produced by epidermal cells, and thus serves as an inhibitor of expression of IL-33, IL-1a, and IL-1β gene.

The compound represented by formula (I) above can be synthesized by, for example, the following synthetic method described in JP 2011-079772 A.

### Intermediate: Synthesis of 2-O-Glyceryl Ascorbic Acid

L-ascorbic acid and sodium hydrogen carbonate are added to water in an argon atmosphere and stirred at room temperature for 30 minutes, followed by addition of glycidol. The mixture is heated to 60° C. and stirred for 5 hours. Methanol is added to the mixture and then filtered. The filtrate is condensed under reduced pressure. The thus obtained residue is subjected to a silica gel column chromatography. The residue is eluted with chloroform/methanol/water=6/4/1 and condensed under reduced pressure, to obtain 2-O-glyceryl ascorbic acid.

### Synthesis of 2-O-Glyceryl-6-O-Hexadecanoyl Ascorbic Acid

Pyridine is added to the synthesized 2-O-glyceryl ascorbic acid in an argon atmosphere, followed by addition of n-hexadecanoic acid anhydride, and stirred at 60° C. for 3 hours. Thereafter, water is added to the mixture and extracted with ethyl acetate. The extraction liquid is condensed under reduced pressure, and 134 mg of the obtained residue is subjected to a silica gel column chromatography. The residue is eluted with a chloroform/methanol/water=7/3/0.3 mixed solution, and then purified and condensed under reduced pressure, to obtain 2-O-glyceryl-6-O-hexadecanoyl ascorbic acid.

In the preparation of this embodiment, the concentration of the compound represented by formula (I) above can be, for example, 0.001 mass % or more and 5.0 mass % or less, and is preferably 0.01 mass % or more and 2.0 mass % or less. An advantage of the aforementioned concentration is to enable further improvement of atopic dermatitis.

The preparation generally includes water and can further include, for example, a thickener, a surfactant, and a preservative, other than the aforementioned components.

The preparation of this embodiment is not particularly limited to a specific form, but is, for example, a liquid form. The preparation of this embodiment can be a solid form.

The preparation of this embodiment can be produced by a general method. The preparation can be produced by, for example, mixing the components to be compounded and stirring the mixture. A general stirrer can be used as a device for stirring. Stirring can be performed while heating, as needed.

The preparation is preferably a skin external preparation (percutaneous administration agent) or cosmetics. Such a skin external preparation or cosmetics are generally applied to skin for use. The skin external preparation or cosmetics can be applied to, for example, facial skin, neck skin, skin of the extremities, head skin, hair, or mucous membrane in nares, lips, ears, genitals, or anus for use. The skin external preparation or cosmetics can be used as a bathing agent, or can be used as an adhesive skin patch.

The preparation of this embodiment is not necessarily restricted to the category such as cosmetics, quasi drugs, or medications set forth in Pharmaceutical Affairs Law.

The atopic dermatitis improving agent, the interleukin production inhibitor, and the inhibitor of expression of interleukin gene of the present invention have been described above for example by way of embodiment, without limitation thereto. In the present invention, various forms that can be adopted for a general composition for external skin and a general composition for oral administration can be adopted as long as the effects of the present invention are not impaired.

The matters disclosed herein include the following.

(1)

An atopic dermatitis improving agent including a glyceryl ascorbic acid acylated derivative represented by formula (I) below.

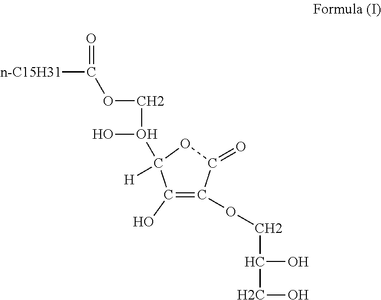

(2)

An interleukin production inhibitor including the glyceryl ascorbic acid acylated derivative represented by formula (I) above.

(3)

An inhibitor of expression of interleukin gene, which includes the glyceryl ascorbic acid acylated derivative represented by formula (I) above.

(4)

Use of the glyceryl ascorbic acid acylated derivative represented by formula (I) above for improving atopic dermatitis.

(5)

Use of the glyceryl ascorbic acid acylated derivative represented by formula (I) above for inhibiting the production of interleukin.

(6)

Use of the glyceryl ascorbic acid acylated derivative represented by formula (I) above for inhibiting expression of interleukin gene.

(7)

Method for treating atopic dermatitis by applying a preparation including the glyceryl ascorbic acid acylated derivative represented by formula (I) above to the skin that has developed atopic dermatitis.

### EXAMPLES

Next, the present invention will be described in more detail by way of Examples, without limitation thereto.

### Test Example 1

A preparation of each of test examples (usable as, for example, a skin external preparation) was produce by dissolving 2-O-glyceryl-6-O-hexadecanoyl ascorbic acid (i.e., compound represented by formula (I)) in a solvent such as water. The concentration was set at, for example, 10 μM, and 20 μM in each test as appropriate.

### Test Example 2

A preparation was produced in the same manner as in Test Example 1 except that 2-O-glyceryl-6-dodecanoyl ascorbic acid was used instead of the compound represented by formula (I).

### Test Example 3

A preparation was produced in the same manner as in Test Example 1 except that 2-O-glyceryl-6-tetradecanoyl ascorbic acid was used instead of the compound represented by formula (I).

### Test Example 4

A preparation was produced in the same manner as in Test Example 1 except that 2-O-glyceryl-6-octadecanoyl ascorbic acid was used instead of the compound represented by formula (I).

### Test Example 5

A preparation was produced in the same manner as in Test Example 1 except that ascorbic acid was used instead of the compound represented by formula (I).

### Test Example 6

A preparation was produced in the same manner as in Test Example 1 except that ascorbyl palmitate was used instead of the compound represented by formula (I).

### <Evaluation Regarding the Inhibition of Cytokine Production (In Vitro Tests)>

The following provides the details of in vitro tests. 6×10⁴ cells of Normal Human Epidermal Keratinocytes (NHEK manufactured by KURABO INDUSTRIES LTD.) were seeded on a 12 well plate and cultured at 37° C. under 5% CO₂ conditions for 72 hours. The incubation medium was removed and then replaced by a culture medium including the following inflammatory stimulating substance (final concentration: 25 ng/mL) and the individual compound used in each of the above Test Examples, followed by further culturing at 37° C. under 5% CO₂ conditions for 3 hours. Thereafter, total RNA was purified from cells according to the instruction manual of product name “PureLink RNA Mini kit” (INVITROGEN), and RNA purified using the test kit “ReverTra Ace™ qPCR RT Master Mix (manufactured by TOYOBO CO., LTD.)” was reverse-transcribed to cDNA. For the cDNA, IL-33 mRNA expression level was quantitatively evaluated by a real time PCR method. Glyceraldehyde-3-phosphate dehydrogenase (GAPDH) was used as an internal standard calibration. The compounds used in Test Examples were dissolved in dimethyl sulfoxide (DMSO) to prepare the individual stocks, and then respectively dissolved in the culture media to have a concentration of 10 to 20 μM. At this time, dilution was performed to allow DMSO to have a concentration (1% or less) at which no cytotoxicity is caused.

### Inflammatory Stimulating Substance (Phorbol12-Myristate13-Acetate) PMA

Negative control (NC/No PMA added)
Positive control (Steroidal anti-inflammatory drugs) (Dexamethasone) DEX, concentration: 10 μM

Evaluations for interleukin-1α (IL-1α) and interleukin-1β (IL-1β) were performed in the same manner as above.

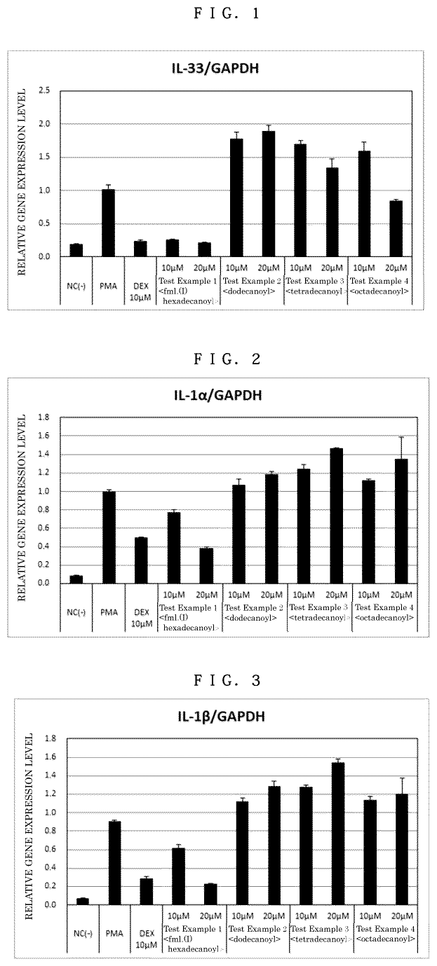

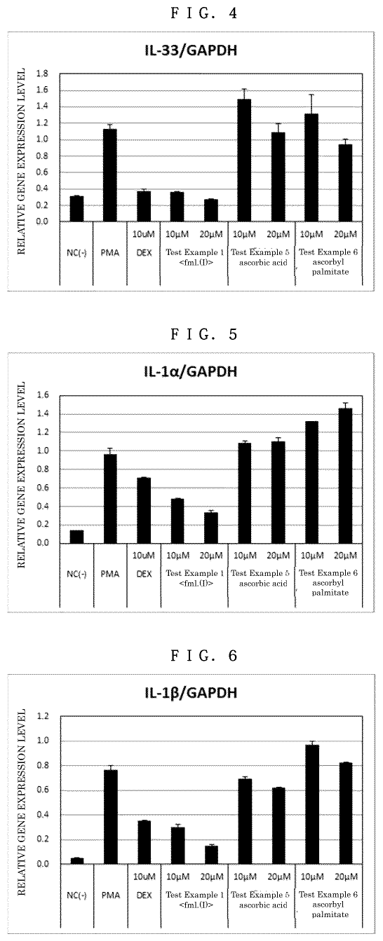

For Test Examples 1 to 4, graphs showing the results of in vitro tests for the above evaluation regarding the inhibition of cytokine production are shown in FIG. 1 to FIG. 3. For Test Examples 1, 5, and 6, graphs showing the results of in vitro tests for the above evaluation regarding the inhibition of cytokine production are shown in FIG. 4 to FIG. 6. As can be seen from FIG. 1 to FIG. 6, expression of cytokine gene strongly related to exacerbation of atopic dermatitis was inhibited by the preparations including 2-O-glyceryl-6-O-hexadecanoyl ascorbic acid. Accordingly, the atopic dermatitis improving agent (the interleukin production inhibitor, and the inhibitor of expression of interleukin gene) of this embodiment can improve atopic dermatitis.

### INDUSTRIAL APPLICABILITY

The atopic dermatitis improving agent of the present invention can be used as, for example, a skin external preparation or cosmetics. The interleukin production inhibitor and the inhibitor of expression of interleukin gene of the present invention can be used by being applied to the skin for the purpose of, for example, preventing and reducing of dry skin caused by atopic dermatitis, or preventing and reducing of inflammatory skin disorder caused by atopic dermatitis. The atopic dermatitis improving agent and the like of the present invention can be appropriately used as medications, quasi drugs, or cosmetics, by being directly applied to, for example, the stratum corneum.

1. An atopic dermatitis improving agent comprising a glyceryl ascorbic acid acylated derivative represented by formula (I) below.

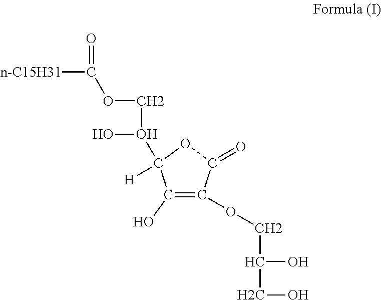

2. An interleukin production inhibitor comprising a glyceryl ascorbic acid acylated derivative represented by formula (I) below and being configured to inhibit production of interleukin-33 and interleukin-1.

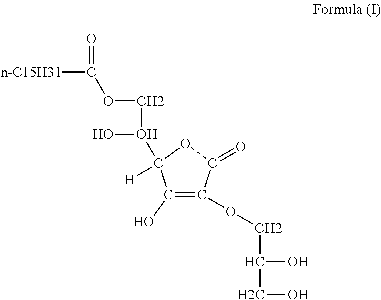

3. An inhibitor of expression of interleukin gene, the inhibitor comprising a glyceryl ascorbic acid acylated derivative represented by formula (I) below and being configured to inhibit expression of interleukin-33 gene and interleukin-1 gene. [chemical structure]
